(19) United States
(12) Patent Application Publication
(10) Pub. No.: US 2024/0327331 A1
(43) Pub. Date: Oct. 3, 2024

(54) ESTERS OF 7-COOH CBD DERIVATIVES AND THEIR USE AS PREVENTION, PROPHYLAXIS OF PROGRESSION, AND/OR TREATMENT OF NEUROGENERATIVE DISEASES

(71) Applicant: Receptor Pharma INC, Dover, DE (US)

(72) Inventors: Marguérite Mensonides-Harsema, Malmö (SE); Zacharia Iskander RESSAISSI, Lomma (SE); Charlott Brunmark, Flyinge (SE); Karin von Wachenfeldt, Lund (SE)

(73) Assignee: Receptor Pharma INC, Dover, DE (US)

(21) Appl. No.: 18/579,469

(22) PCT Filed: Jun. 20, 2022
(86) PCT No.: PCT/EP2022/066656

§ 371 Date: Jan. 15, 2024

(30) Foreign Application Priority Data
Jul. 13, 2021 (SE) 2150937-7

(51) Int. Cl. C07C 69/757; A61K 31/00; A61P 25/28; C07D 207/38; C07D 295/03

(52) U.S. Cl. CPC C07C 69/757; A61K 31/658; A61P 25/28; C07D 207/38; C07D 295/03; C07C 2601/16

(57) ABSTRACT

The present invention relates to compound of formula (I), a diastereomer, enantiomer, crystal or co-crystal thereof, or a pharmaceutically acceptable salt, or mixture thereof. The present invention also relates to pharmaceutical compositions comprising these compounds and to a process of preparing said compounds as well as to their use as medicaments for the prevention, prophylaxis of progression, and/or treatment of a disease in which an antagonistic effect of the G-protein coupled receptor 3 (GPR3) is beneficial, such as neurogenerative diseases, like dementias, including Alzheimer disease.

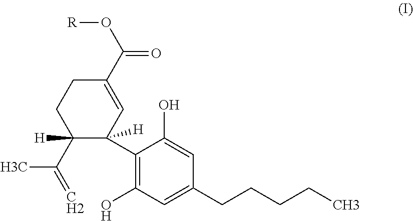

### FIELD OF THE INVENTION

The present invention relates to new compounds of the general formula (I) and pharmaceutically acceptable salts and/or co-crystals thereof, which are pro-drugs of 7-Nor-7-carboxy-cannabidiol and to a process of preparing said compounds. The present invention also relates to pharmaceutical compositions comprising these compounds and to their use as medicaments for the prevention, prophylaxis of progression, and/or treatment of a disease in which an antagonistic effect of the G-protein coupled receptor 3 (GPR3) is beneficial, such as neurogenerative diseases, like dementias, including Alzheimer disease.

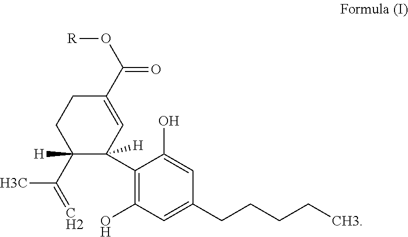

### BACKGROUND

Neurodegenerative diseases are those that affect neurons. The degenerative process can involve the progressive loss of neuronal structure, the progressive loss of neuronal function, or progressive neuron cell death. Such progressive neurodegeneration often results in physical disability and mental deterioration. Many neurodegenerative diseases are severely progressive and unremitting, and there are few, if any, curative treatments. Neurodegenerative diseases, like Alzheimer—(AD), Parkinson's—(PD), Lewy Body—(LBD), Lou Gehrig's—(ALS), Huntingdon's disease, and frontotemporal dementia (FTD), affect a large percentage of the global population. Because neurodegenerative diseases strike primarily in mid- to late life, the incidence is expected to soar as the global population ages. The general prevalence of AD, for example, is 1 in 300, and affects 1-2% of the over 65 years of age population. The first clinical presentation of AD is characterized by loss of memory, cognition, reasoning, judgement, and orientation. In a second stage of the disease, motor, sensory and linguistic abilities are lost, leading to severe impairment and ultimately death. The time between clinical onset and diagnosis and death ranges between 4 and 12 years. In the brain of AD patients, two major pathologic processes are discerned. Firstly, the high level of neurofibrillary tangles which are aggregates of hyperphosphorylated Tau protein—a protein that is particularly abundant in the axons of neurons and that is normally associated with the intracellular microtubules. Microtubules are an important component of the cytoskeleton, regulating for example vesicular transport and cytokinesis. Secondly, deposits of beta-amyloid plaques in both the brain and cerebral blood vessels. These protein tangles and peptide plaques are believed to be neurotoxic as well as pro-inflammatory.

There are to date no treatments available that can cure neurodegenerative diseases, including AD. Thus, today's patients are treated with pharmaceuticals that rather target the symptoms of these diseases and/or aim to slow their progression.

Cannabinoids are known to treat a wide range of health conditions. Thirty-three states of the United States and the District of Colombia of the United States enacted medical marijuana laws that recognize that cannabinoids can treat over 100 different conditions including acquired immune deficiency syndrome (“AIDS”), Alzheimer's disease, amyotrophic lateral sclerosis (“ALS”), anorexia, anxiety, Arnold-Chiari malformation, arthritis, autism, autism spectrum disorder, cachexia, cancer, causalgia, cerebral palsy, chemotherapy-induced anorexia, chronic is back pain, chronic pain, chronic seizures, chronic traumatic encephalopathy, complex regional pain syndrome, complex regional pain syndrome type II, cramping, Crohn's disease, cystic fibrosis, debilitating psychiatric disorders, decompensated cirrhosis, diabetes mellitus, Dravet syndrome, dyskinetic movement disorders, dystonia, Ehlers-Danlos syndrome, elevated intraocular pressure, epidermolysis bullosa, epilepsy, fibromyalgia, fibrous dysplasia, glaucoma, headache, hepatitis C, Huntington's disease, hydrocephalus, hydromyelia, idiopathic pulmonary fibrosis, inclusion body myositis, inflammatory autoimmune-mediated arthritis, inflammatory bowel disease, inflammatory demyelinating polyneuropathy, interstitial cystitis, intractable epilepsy, intractable pain, intractable skeletal muscular spasticity, intractable spasticity, Lennox-Gastaut syndrome, lupus, migraines, mitochondrial disease, multiple sclerosis, muscle spasms, muscular dystrophy, myasthenia gravis, myoclonus, nail-patella syndrome, neural-tube defects, neurodegenerative diseases, neurofibromatosis, neurological disorders, neuropathic pain, neuropathy, obstructive sleep apnea, osteogenesis imperfecta, pancreatitis, Parkinson's disease, peripheral neuropathy, post herpetic neuralgia, post laminectomy syndrome, post-concussion syndrome, post-traumatic stress disorder (“PTSD”), psoriasis, psoriatic arthritis, reflex sympathetic dystrophy, renal failure requiring dialysis, residual limb pain, rheumatoid arthritis, seizure disorders, sickle cell anemia, sickle cell disease, Sjogren's syndrome, spasmodic torticollis (cervical dystonia), spasticity, spinal cord disease, spinal cord injury, spinal stenosis, spinocerebellar ataxia, syringomyelia, Tarlov cysts, Tourette's syndrome, traumatic brain injury (“TBI”), ulcerative colitis, and visceral pain.

Cannabidiol (CBD; CAS 13956-29-1; Formula II) is one of the naturally occurring, non-psychotropic cannabinoids, that is found in up to 40% in the extract of the cannabis plant. In its purified form, it has shown promise as an analgesic, anticonvulsant, muscle relaxant, anxiolytic, and antipsychotic among currently investigated uses, as it has shown neuroprotective, anti-inflammatory, and antioxidant activity. CBD interacts with several cell membrane bound receptors, enzymes, and transporters. It's target receptors include G-protein coupled receptors like the cannabinoid receptors C1 and C2 (CB1, CB2), the serotonin receptors 5HT1A, 5HT2A and 5HT3A, the orphan receptors GPR 3, 6, 12, 18 and 55, as well as the glycine receptors α1, α1/β and α3, acetylcholine subunit α7, the nuclear receptor PPAR6, the prostaglandin receptors PTGS1 and 2, the adenosine receptor A1, the calcium ion channels 1G, 1H and 11, the opioid receptors δ and μ, the ion channels TRPV 1, 2, 3 and 4, the ion channels TRP A1 and M8. It also interacts with the transporter proteins ABCC1 (MDR1) and ABCG2 (BCRP) and with the CYP enzymes 1A1, 1A2, 1B1, 2C9, 2C19, 2D6, 3A4, 3A5, 3A7 and 17A1.

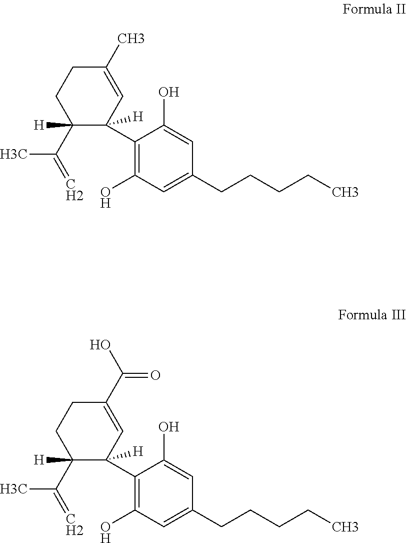

Following the expression and embedding of the G-protein coupled receptors in the membrane of a cell, its life cycle describes four phases: ligand binding (endogenous or exogenous), intracellular coupling to a G-protein, desensitization through recruitment and interaction with β-arrestins and recycling. Anomalies in the activity of GPCRs are implicated in the onset and is progression of many diseases, including cancers, cardiovascular-, metabolic- and mental disorders, as well as neurodegenerative diseases. Up to a third of the pharmaceutical treatments exert their effect through the modulation (agonism, antagonism or inverse agonism) of one or several GPCRs. GPRs 3, 6, 12, 18 and 55 are called orphan receptors, because there are no confirmed endogenous agonists known for them. The GPR3 and GPR 6 receptors are constitutively active receptors. They are expressed in the central nervous system. The GPR3 receptor is involved in both neuron differentiation and maturation. Modulation of the GPR3 receptor activity alters emotional behaviors, is involved in the development of neuropathic pain and it has been suggested for both receptors that they play a role in Alzheimer's disease, in particular the formation of beta amyloid plaques, with the GPR6 receptor playing a neuroprotective role while the GPR3 receptor promotes the production of amyloid-R. Lee et al. (2001) named the GPR3 and GPR6 receptors cannabinoid receptor-like orphan GPCRs. This prompted Laun and Song (2017) to study the interaction of various phyto- and endocannibinoids with these two receptors. None of the test compounds showed any significant effect at the GPR3 or GPR6 receptor, except for CBD. They showed that CBD, like N-[1(S)-[4-[[4-methoxy-2-[(4-methoxyphenyl)sulfonyl]phenyl]-sulfonyl]phenyl]ethyl]methanesulfonamide (Sch.336; Lunn et al 2006), acted as an inverse agonist at the GPR3 receptor, dose-dependently inhibiting its activity by 39%, with and IC50 of 1.22 μM.

CBD, as Epidiolex, is currently approved to treat the orphan diseases Lennox-Gastaut syndrome and Dravet syndrome. It is believed to exert its anticonvulsant effects through modulation of the GPR55 and adenosine signaling system. In the body, CBD is converted to 2 major metabolites. Metabolic oxidation of CBD generates the first major metabolite 7-hydroxy-CBD (7-OH-CBD). The plasma levels of the 7-OH metabolite are about 50% of the plasma levels of the parent compound CBD. Like its parent compound, 7-OH-CBD acts as an anticonvulsant. In fact, 7-OH-CBD shows equipotency to CBD in animal models of epilepsy. The second major metabolite, 7-carboxy-CBD (7-Nor-7-carboxy-cannabidiol; (3R-trans)-3-(2,6-dihydroxy-4-pentylphenyl)-4-(1-methylethenyl)-1-cyclohexene-1-carboxylic acid; 7-COOH-CBD; (3R-trans)-cannabidiol-11-oic acid; CAS 63958-77-0; Formula III) is formed through metabolic oxidation of 7-OH-CBD. The 7-COOH-CBD metabolite circulates in the plasma at levels up to 50-fold of that of the parent compound CBD. However, it is devoid of any anticonvulsant action and showed no binding or modulation properties at physiological relevant concentrations in an in vitro binding/target panel that included CB1 and CB2 receptors as well as the voltage gated Na channel and mono-amine transporters (FDA non-clinical review of Epidiolex).

WO99/53917 discloses cannabinoids (CBS) derivatives for use as radical scavengers. Although no binding data are shown, these compounds are supposed to be weak agonists of both the NMDA and the CB receptor. The most preferred compounds are tricyclic CBS derivatives. Some of the CBS derivatives are substituted with one or two carboxylic acid groups. 7-COOH-CBD is mentioned as one of the CBS derivatives and as being a weak agonist of both the NMDA and the CB receptor. WO99/53917 does not mention the antagonistic properties of 7-COOH-CBD on the GPR3 receptor. WO99/53917 is also silent about esterification of CBD derivatives.

Tayler M., Advanced Drug Delivery Reviews, 19 (1996), p 131-148, Improved passive oral drug delivery via prodrugs, mentions esterification of drugs in general. It is clear from Tayler that preparing an ester of a known compound is complex and does not automatically mean an improvement in bioavailability of the parent compound. Tayler explains that several physicochemical parameters for prodrugs must be considered in preparation of a prodrug, such as water and lipid solubility, partition coefficient, hydrogen bonding and desolvation properties, and molecular size. The relationship of chemical structure and physicochemical properties to permeability is more complex than solubility. Increasing the size of a molecule will decrease the rate of diffusion through the membrane. Taylor is silent about esterification of CBD or its derivatives.

There is still a need for compounds that have an antagonistic effect on the GPR3 receptor. There is still a need for compounds that have an improved potency and improved selectivity for and/or that do not act as agonists or inverse agonists at the G-protein coupled receptor 3 (GPR3). There is a need for compounds having reduced side effect, such as gastrointestinal toxicity. There is a need for compounds that modulate or reduce the amount of Tau protein in a cell and/or the amount of neurofibrillary tangles and/or the amount of the peptide fragment amyloid beta and/or the amount of beta amyloid plaques in the brain and/or in cerebral blood vessels. There is a need for effective and efficient prevention and/or treatment of neurogenerative diseases, like dementias, including Alzheimer disease.

### SUMMARY OF THE INVENTION

The present invention relates to a compound of formula I, a diastereomer, enantiomer, crystal or co-crystal thereof, hydrates, solvates, or a pharmaceutically acceptable salt, or mixture thereof

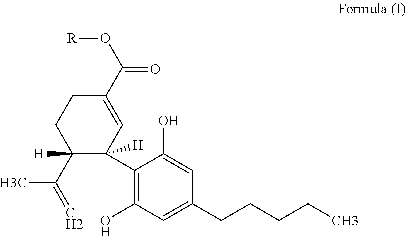

wherein R is selected from the group comprising or consisting of C₁₋₆alkyl, C₃₋₆alkenyl, C₁₋₆heteroalkyl, C₃₋₆heteroalkenyl, C₃₋₆cycloalkyl, C₃₋₆cycloalkenyl, C₃₋₆heterocycloalkyl, C₃₋₆heterocycloalkenyl, C₃₋₆heterocycloalkenylC₁₋₆alkyl, C₅₋₆aryl, C₅₋₆heteroaryl, C₃₋₆cycloalkylC₁₋₄alkyl, C₃₋₆cycloalkylC₃₋₄alkenyl, C₃₋₆heterocycloalkylC₁₋₄alkyl, C₃₋₆heterocycloalkylC₃₋₄alkenyl, C₅₋₆arylC₁₋₄alkyl, C₅₋₆arylC₃₋₄alkenyl, C₅₋₆heteroarylC₁₋₄alkyl, and C₃₋₆heteroarylC₃₋₄alkenyl which are optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl.

The GPR3 receptor is constitutively active. CBD acts as an inverse agonist at the GPR3 receptor, inducing an altered pharmacological response from the natural effects of the GPR3 receptor's activity. Surprisingly, we have now found that 7-COOH-CBD does not act as an inverse agonist at the GPR3 receptor, like CBD. 7-COOH-CBD acts as an antagonist, effectively dampening the pathological overactivity of GPR3 receptors. 7-COOH-CBD is not expected to be psychoactive nor have any anticonvulsant effects. It is also believed that the novel esters of the invention have an improved bioavailability and/or tolerability compared to 7-COOH-CBD.

The medication would be taken chronically and thus the carboxylic acid function may cause ulcers in the upper gastro-intestinal tract with long-term use. Also, from a pharmacokinetic/ADME perspective, the dose/exposure ratio of the novel esters of the invention is improved. For example, we could protect the stomach by using formulations (pH-dependent modified release technologies) but then any absorption in the upper gastro-intestinal tract is lost. Besides, tablets can stay behind in the stomach, especially in elderly, and when the tablets are then finally reaching the small intestines with the higher pH, the amount of drug released may be too high.

Another advantage is related to a physicochemical/formulation of carboxylic compounds. The carboxylic acid is amorphous, and the melting point is “stretched”/sub-optimal for tabletting purposes.

However, the carboxylic acid group of 7-COOH-CBD presents a disadvantage in a drug substance meant for the chronic, long-term treatment of a disease. First, drug substances with a carboxylic acid group are known to induce peptic ulcer disease and upper gastro-intestinal bleeding, with the best-known examples being aspirin and non-steroidal anti-inflammatory drugs. This risk may be somewhat reduced by developing special formulations that release the drug slowly into the GI tract and/or in the lower parts of the GI tract that are less acidic. However, the carboxylic acid group in these more basic environments will be deprotonated, which prevents the absorption of the compound through the gut wall into the blood stream. In the novel compounds of formula (I) disclosed in the present invention, the carboxylic acid group is transformed into an ester. Esterases that are present in the blood stream remove the ester group, transforming the compounds of formula I into 7-COOH-CBD. Thus, drug products containing a compound of formula I are readily available for absorption in the GI tract to form the active metabolite 7-COOH-CBD. Making esters of 7-COOH-CBD is not straight forward due to the presence of other hydroxy groups on the compounds. This problem has been overcome by the specific process for preparing the compounds.

The novel compounds are believed to have an improved potency and selectivity for the modulation of GPR3 receptor. It is believed that the compounds of the invention would have a reduced potential for side effects associated with conventional antagonists for GPR3 receptor. The compounds of the invention are believed to have a reduced gastrointestinal toxicity.

In some aspects, R is selected from the group comprising or consisting of C₁₋₆alkyl, C₃₋₆alkenyl, C₃₋₆cycloalkyl, C₃₋₆cycloalkenyl, C₃₋₆heterocycloalkylC₁₋₆alkyl, C₃₋₆heterocycloalkenylC₁₋₆alkyl, C₅₋₆arylC₁₋₄alkyl and C₃₋₆heteroarylC₁₋₄alkyl, which are optionally substituted with one or more R¹, wherein R¹ is independently selected from the group comprising or consisting of halogen, —OH, —CF₃, ═O, —NO₂, —C(O)NR²R³, —COOR², R², —OR², and —NR²R³, wherein R² and R³ are independently selected from the group comprising or consisting of hydrogen and C₁₋₆alkyl.

In some aspects, R is selected from the group comprising or consisting of C₁₋₄alkyl, C₃₋₅alkenyl, C₃₋₆cycloalkyl, C₃₋₆cycloalkenyl, C₄₋₆heterocycloalkylC₁₋₃alkyl, C₄₋₆heterocycloalkenylC₁₋₃alkyl and C₅₋₆arylC₁₋₃alkyl, which are optionally substituted with one or more R¹, wherein R¹ is independently selected from the group comprising or consisting of hydrogen, chloro, fluoro, —OH, —CF₃, ═O, C₁₋₃alkyl, —OC₁₋₃alkyl, and —NH₂.

In some aspects, R is selected from the group comprising or consisting of methyl, ethyl, propyl, isopropyl, propenyl, butyl, isobutyl, tertbutyl, butenyl, pentyl, pentenyl, cyclopropyl, cyclobutyl, cyclopentyl, cyclopentenyl, piperidinyl, piperidinomethyl, piperazinyl, piperazinylmethyl, piperazinylethyl, phenyl, phenylmethyl, phenylethyl, pyrrolinyl, pyrrolinylmethyl, pyrrolinylethyl, pyrrolinyl, pyrrolinylmethyl and pyrrolinylethyl, and R¹ is independently selected from the group comprising or consisting of fluoro, chloro, ═O, C(O)NR²R³, —COOR², R², —OR², and —NR²R³, wherein R² and R³ are independently selected from the group comprising or is consisting of hydrogen and C₁₋₃alkyl.

In some aspects, R is selected from the group comprising or consisting of methyl, ethyl, propyl, butyl, pentyl, hexyl, cyanomethyl, methoxymethyl, 2-oxoethyl, 2-aminoethyl, 2-cyanoethyl, 1-methylethyl, 2-methylethyl, 1,1-dimethylethyl, 2-cyano-1-methylethyl, 2,2-difluoroethyl, 2,2,2-trifluoroethyl, 2-(dimethylamino)ethyl, 2-amino-2-ethoxyethyl, 1-methylpropyl, 2-methylpropyl, 3-aminopropyl, 1,1-dimethylpropyl, 2,2-dimethylpropyl, 2-cyanopropyl, 3-(ethenyloxy)propyl, 3-oxobutyl, 1-methylbutyl, 2-methylbutyl, 3-methylbutyl, 1,1-dimethylbutyl, 2-propyn-1-yl, 2-propen-1-yl, 1-methyl-2-propyn-yl, 2-methyl-2-propen-1-yl, 3-buten-1-yl, 2-amino-1-methylethyl, 1-cyclopenten-1-yl, 3-cyclopenten-1-yl, cyclopropyl, 1-(hydroxymethyl)cyclopropyl, cyclobutyl, 3-hydroxy-cyclobutyl, 3-methoxy-cyclobutyl, 1-cyanocyclobutyl, cyclopentyl, 1-methylcyclopentyl, 3-methylcyclopentyl, 2-hydroxycyclopentyl, 3-hydroxycyclopentyl, 2,3-dihydroxycyclopentyl, cyclohexyl, 1Hpyrrol-2-yl, 1H-pyrrol-3-yl, 3-pyrrolidinyl, tetrahydro-3-furanyl, 3-pyridinyl, 2-thienyl, 1H-pyrrol-2-ylmethyl, 1H-pyrrol-3-ylmethyl, 2-furanylmethyl, 1H-imidazol-2-yl, 1-methyl-1H-pyrazol-3-yl, 5-methyl-2-furanyl, 1-cyclohexen-1-yl, 4-methyl-4H-1,2,4-triazol-3-yl, 1H-1,2,4-triazol-5-ylmethyl, 2-thiazolyl, 4-piperidinyl, 1-methyl-3-pyrrolidinyl, 1,3,4-thiadiazol-2-yl, phenyl, cyclopropylmethyl, cyclobutylmethyl, cyclopentylmethyl, (1-hydroxycyclobutyl)methyl, (1-hydroxycyclobutyl)methyl, (3-hydroxycyclobutyl)methyl, (3-methoxycyclobutyl)-methyl, (tetrahydro-2-furanyl)methyl, (tetrahydro-3-furanyl)methyl, 2-cyclopropylethyl, 1-cyclobutylethyl, 2-cyclobutylethyl, chlorophenyl, p-chlorophenylmethyl, metoxyphenyl, o-methoxyphenylmethyl, 2-aminocyclopentenyl, 2-fluorocyclopentenyl, piperidinomethyl, pyrrolinyl, pyrrolinylmethyl, pyrrolinylethyl, oxopyrrolinyl, oxopyrrolinylmethyl, piperazinyl, 1-piperazinyl)methyl, 4-methyl-1-piperazinyl)methyl and oxopyrrolinylethyl.

In some aspects, R is selected from the group comprising or consisting of methyl, ethyl, propyl, isopropyl, n-butyl, 3-butenyl, 1-methylpropyl, isobutyl, tertbutyl, cyclopropane, cyclobutane, cyclopentane, 1-cyclopentene, 3-cyclopentene, 2-amino-1-cyclopentene, 2-fluoro-1-cyclopentene, piperidinomethyl, 1-piperazinylmethyl, (4-methyl-1-piperazinyl)methyl, (p-chlorophenyl)methyl, (o-methoxyphenyl)methyl, and (5-oxo-3pyrrolin-2-yl)methyl.

In some aspects, R is selected from the group comprising or consisting of methyl, ethyl, propyl, isopropyl, n-butyl, 3-butenyl, 1-methylpropyl, isobutyl, tertbutyl, cyclopropane, and cyclobutene.

In some aspects, R is selected from the group comprising or consisting of cyclopentane, 1-cyclopentene, 3-cyclopentene, 2-amino-1-cyclopentene, and 2-fluoro-1-cyclopentene.

In some aspects, R is selected from the group comprising or consisting of piperidinomethyl, 1-piperazinylmethyl, (4-methyl-1-piperazinyl)methyl, (p-chlorophenyl)methyl, (o-methoxyphenyl)methyl, and (5-oxo-3pyrrolin-2-yl)methyl.

The invention also relates to compounds selected from the group comprising or consisting of
(3R-trans)-3-(2,6-dihydroxy-4-pentylphenyl)-4-(1-methylethenyl)-1-cyclohexene-1-carboxylic acid methyl ester,
(3R-trans)-3-(2,6-dihydroxy-4-pentylphenyl)-4-(1-methylethenyl)-1-cyclohexene-1-carboxylic acid ethyl ester,
(3R-trans)-3-(2,6-dihydroxy-4-pentylphenyl)-4-(1-methylethenyl)-1-cyclohexene-1-carboxylic acid propyl ester,
(3R-trans)-3-(2,6-dihydroxy-4-pentylphenyl)-4-(1-methylethenyl)-1-cyclohexene-1-carboxylic acid isopropyl ester,
(3R-trans)-3-(2,6-dihydroxy-4-pentylphenyl)-4-(1-methylethenyl)-1-cyclohexene-1-carboxylic acid n-butyl ester,
(3R-trans)-3-(2,6-dihydroxy-4-pentylphenyl)-4-(1-methylethenyl)-1-cyclohexene-1-carboxylic acid 3-butenyl ester,
(3R-trans)-3-(2,6-dihydroxy-4-pentylphenyl)-4-(1-methylethenyl)-1-cyclohexene-1-carboxylic acid 1-methylpropyl ester,
(3R-trans)-3-(2,6-dihydroxy-4-pentylphenyl)-4-(1-methylethenyl)-1-cyclohexene-1-carboxylic acid isobutyl ester,
(3R-trans)-3-(2,6-dihydroxy-4-pentylphenyl)-4-(1-methylethenyl)-1-cyclohexene-1-carboxylic acid tertbutyl ester,
(3R-trans)-3-(2,6-dihydroxy-4-pentylphenyl)-4-(1-methylethenyl)-1-cyclohexene-1-carboxylic acid cyclopropane ester,
(3R-trans)-3-(2,6-dihydroxy-4-pentylphenyl)-4-(1-methylethenyl)-1-cyclohexene-1-carboxylic acid cyclobutane ester,
(3R-trans)-3-(2,6-dihydroxy-4-pentylphenyl)-4-(1-methylethenyl)-1-cyclohexene-1-carboxylic acid cyclopentane ester,
(3R-trans)-3-(2,6-dihydroxy-4-pentylphenyl)-4-(1-methylethenyl)-1-cyclohexene-1-carboxylic acid 1-cyclopentene ester,
(3R-trans)-3-(2,6-dihydroxy-4-pentylphenyl)-4-(1-methylethenyl)-1-cyclohexene-1-carboxylic acid 3-cyclopentene ester,
(3R-trans)-3-(2,6-dihydroxy-4-pentylphenyl)-4-(1-methylethenyl)-1-cyclohexene-1-carboxylic acid 2-amino-1-cyclopentene ester,
(3R-trans)-3-(2,6-dihydroxy-4-pentylphenyl)-4-(1-methylethenyl)-1-cyclohexene-1-carboxylic acid 2-fluoro-1-cyclopentene ester,
(3R-trans)-3-(2,6-dihydroxy-4-pentylphenyl)-4-(1-methylethenyl)-1-cyclohexene-1-carboxylic acid piperidinomethyl ester,
(3R-trans)-3-(2,6-dihydroxy-4-pentylphenyl)-4-(1-methylethenyl)-1-cyclohexene-1-carboxylic acid 1-piperazinylmethyl ester,
(3R-trans)-3-(2,6-dihydroxy-4-pentylphenyl)-4-(1-methylethenyl)-1-cyclohexene-1-carboxylic acid (4-methyl-1-piperazinyl)methyl ester,
(3R-trans)-3-(2,6-dihydroxy-4-pentylphenyl)-4-(1-methylethenyl)-1-cyclohexene-1-carboxylic acid (p-chlorophenyl)methyl ester,
(3R-trans)-3-(2,6-dihydroxy-4-pentylphenyl)-4-(1-methylethenyl)-1-cyclohexene-1-carboxylic acid (o-methoxyphenyl)methyl ester, and
(3R-trans)-3-(2,6-dihydroxy-4-pentylphenyl)-4-(1-methylethenyl)-1-cyclohexene-1-carboxylic acid (5-oxo-3pyrrolin-2-yl)methyl ester,
or a diastereomer, enantiomer, crystal or co-crystal thereof, hydrates, solvates, or a pharmaceutically acceptable salt, or mixture thereof.
The invention also relates to compounds selected from the group comprising or consisting of
(3R-trans)-3-(2,6-dihydroxy-4-pentylphenyl)-4-(1-methylethenyl)-1-cyclohexene-1-carboxylic acid methyl ester,
(3R-trans)-3-(2,6-dihydroxy-4-pentylphenyl)-4-(1-methylethenyl)-1-cyclohexene-1-carboxylic acid ethyl ester,
(3R-trans)-3-(2,6-dihydroxy-4-pentylphenyl)-4-(1-methylethenyl)-1-cyclohexene-1-carboxylic acid propyl ester,
(3R-trans)-3-(2,6-dihydroxy-4-pentylphenyl)-4-(1-methylethenyl)-1-cyclohexene-1-carboxylic acid isopropyl ester,
(3R-trans)-3-(2,6-dihydroxy-4-pentylphenyl)-4-(1-methylethenyl)-1-cyclohexene-1-carboxylic acid n-butyl ester,
(3R-trans)-3-(2,6-dihydroxy-4-pentylphenyl)-4-(1-methylethenyl)-1-cyclohexene-1-carboxylic acid 3-butenyl ester,
(3R-trans)-3-(2,6-dihydroxy-4-pentylphenyl)-4-(1-methylethenyl)-1-cyclohexene-1-carboxylic acid 1-methylpropyl ester,
(3R-trans)-3-(2,6-dihydroxy-4-pentylphenyl)-4-(1-methylethenyl)-1-cyclohexene-1-carboxylic acid isobutyl ester,
(3R-trans)-3-(2,6-dihydroxy-4-pentylphenyl)-4-(1-methylethenyl)-1-cyclohexene-1-carboxylic acid tertbutyl ester,
   or a diastereomer, enantiomer, crystal, or co-crystal thereof, hydrates, solvates, or a pharmaceutically acceptable salt, or mixture thereof.

The selectivity and/or potency can be improved by compounds of formula I, whereby the substituents on the ester are relatively short (i.e. 6 carbon atoms or less). These shorter esters is have the advantage of being easier to formulate due to a better crystal structure compared to longer esters. The relatively shorter 7-COOH-CBD derivatives will also be more hydrophylic than the longer esters. When the esters become too long (i.e. 12 carbon atoms or more), the esters will drive the molecular weight up to un-drug-like values (Lipinski rule of Mw˜500).

Compounds of formula I, whereby R is of C₁₋₆alkyl or C₃₋₆alkenyl, have good binding properties.

The invention also relates to compounds selected from the group comprising or consisting of
(3R-trans)-3-(2,6-dihydroxy-4-pentylphenyl)-4-(1-methylethenyl)-1-cyclohexene-1-carboxylic acid cyclopropane ester,
(3R-trans)-3-(2,6-dihydroxy-4-pentylphenyl)-4-(1-methylethenyl)-1-cyclohexene-1-carboxylic acid cyclobutane ester,
(3R-trans)-3-(2,6-dihydroxy-4-pentylphenyl)-4-(1-methylethenyl)-1-cyclohexene-1-carboxylic acid cyclopentane ester,
(3R-trans)-3-(2,6-dihydroxy-4-pentylphenyl)-4-(1-methylethenyl)-1-cyclohexene-1-carboxylic acid 1-cyclopentene ester,
(3R-trans)-3-(2,6-dihydroxy-4-pentylphenyl)-4-(1-methylethenyl)-1-cyclohexene-1-carboxylic acid 3-cyclopentene ester,
(3R-trans)-3-(2,6-dihydroxy-4-pentylphenyl)-4-(1-methylethenyl)-1-cyclohexene-1-carboxylic acid 2-amino-1-cyclopentene ester,
(3R-trans)-3-(2,6-dihydroxy-4-pentylphenyl)-4-(1-methylethenyl)-1-cyclohexene-1-carboxylic acid 2-fluoro-1-cyclopentene ester,
   or a diastereomer, enantiomer, crystal or co-crystal thereof, hydrates, solvates, or a pharmaceutically acceptable salt, or mixture thereof.

Compounds of formula I, whereby R² is C₃₋₆cycloalkyl or C₃₋₆cycloalkenyl have good binding properties.

The invention also relates to compounds selected from the group comprising or consisting of
(3R-trans)-3-(2,6-dihydroxy-4-pentylphenyl)-4-(1-methylethenyl)-1-cyclohexene-1-carboxylic acid piperidinomethyl ester,
(3R-trans)-3-(2,6-dihydroxy-4-pentylphenyl)-4-(1-methylethenyl)-1-cyclohexene-1-carboxylic acid 1-piperazinylmethyl ester,
(3R-trans)-3-(2,6-dihydroxy-4-pentylphenyl)-4-(1-methylethenyl)-1-cyclohexene-1-carboxylic acid (4-methyl-1-piperazinyl)methyl ester,
(3R-trans)-3-(2,6-dihydroxy-4-pentylphenyl)-4-(1-methylethenyl)-1-cyclohexene-1-carboxylic acid (p-chlorophenyl)methyl ester,
(3R-trans)-3-(2,6-dihydroxy-4-pentylphenyl)-4-(1-methylethenyl)-1-cyclohexene-1-carboxylic acid (o-methoxyphenyl)methyl ester, and
(3R-trans)-3-(2,6-dihydroxy-4-pentylphenyl)-4-(1-methylethenyl)-1-cyclohexene-1-carboxylic acid (5-oxo-3pyrrolin-2yl)methyl ester,
   or a diastereomer, enantiomer, crystal or co-crystal thereof, hydrates, solvates, or a pharmaceutically acceptable salt, or mixture thereof.

The invention relates to a pharmaceutical composition comprising the compound of formula I or the list of compounds, as defined above, in the association with a pharmaceutically acceptable adjuvant, diluent and/or carrier.

The invention relates to a process for the preparation of a pharmaceutical composition which comprises mixing the compound of formula I or the list of compounds, as defined above, with a pharmaceutically acceptable adjuvant, diluent or carrier.

The invention also relates to a process for manufacturing compounds of formula I, comprising or consisting of the following steps
1) reacting R-1-imidazolecarboxylate (R-ImC) with 7-COOH-CBD in a suitable polar solvent at a temperature of 20 to 80° C. for 10 to 40 hours under nitrogen atmosphere, to obtain compounds of formula I.

The invention also relates to a process for manufacturing compounds of formula I, comprising or consisting of the following steps
1a) reacting imidazole and R-chloroformate in a suitable organic solvent to obtain R-ImC, and
1b) reacting R-ImC with 7-COOH-CBD in a suitable polar solvent at a temperature of 20 to 80° C. for 10 to 40 hours under nitrogen atmosphere, to obtain compounds of formula I.

In some aspects of the process, the organic solvent in step 1a) is selected from the group comprising or consisting of dichloromethane, 1,2-dichloromethane, acetonitrile, tetrahydrofuran, 2-methyl tetrahydrofuran and 1,2-dimethoxy ethane. In some aspects, the organic solvent is 2-methyl-tetrahydrofuran.

In some aspects, the temperature in step 1a) is about 0° C. for 30 to 90, or 60 minutes while dropping the R-chloroformate into an imidazole solution, and subsequently holding the reaction mixture at room temperature for 12 to 24, or 16 hours.

In some aspects, the molar ratio of imidazole to R-chloroformate is 2 to 4:1 to 3, or 3:2.

In some aspects, the polar solvent in step 1b) is selected from the group comprising or consisting of acetonitrile, tetrahydrofuran, 2-methyl tetrahydrofuran, N,N-dimethyl formamide, dichloromethane and 1,2 dichloro-ethane, or any mixture thereof.

In some aspects, the molar ratio of 7-COOH-CBD to R-ImC is 0.25 to 2:1 to 3, or 1:2

The compounds of formula (I) are believed to reduce the amount of Tau protein in a cell and/or the amount of neurofibrillary tangles and/or the amount of the peptide fragment amyloid beta and/or the amount of beta amyloid plaques in the brain and/or in cerebral blood vessels.

The invention relates to the compound of formula I or the list of compounds, or the pharmaceutical compositions as defined above, for use in therapy.

The invention relates to the compound of formula I or the list of compounds, or the pharmaceutical compositions as defined above, for use in the prevention, progression prophylaxis and/or treatment of a disease of the central nervous system.

The invention relates to the compound of formula I or the list of compounds, or the pharmaceutical compositions as defined above, for use in the prevention, progression prophylaxis and/or treatment of neurodegenerative diseases.

The invention relates to the compound of formula I or the list of compounds, or the pharmaceutical compositions as defined above, for use in the prevention, progression prophylaxis and/or treatment of diseases selected from the group comprising or consisting of Alzheimer disease (AD), Parkinson's disease (PD), Lewy Body disease (LBD), Lou Gehrig's disease (ALS), Huntingdon's disease, and frontotemporal dementia (FTD).

Another embodiment relates to the compound of formula I, or a pharmaceutically acceptable salt thereof, as defined above, for use in prevention, progression prophylaxis and/or treatment of Alzheimer disease (AD).

An embodiment relates to the compound of formula I, or a pharmaceutically acceptable salt thereof, as defined above, for use in prevention, progression prophylaxis and/or treatment of Dementia.

The invention relates to a method of treating, preventing or reducing the risk of a disease in which modulation GPR3 receptor is beneficial.

The invention relates to a method of treating, preventing or reducing the risk of a disease, disorder or condition in which modulation of the GPR3 receptor is beneficial, which comprises administering to a mammal, such as a human, in need thereof, a therapeutically effective amount of a compound of formula I as defined above or a pharmaceutically acceptable salt thereof.

Another embodiment relates to said method of treating, preventing or reducing the risk of diseases selected from the group comprising or consisting of Alzheimer disease (AD), Parkinson's disease (PD), Lewy Body disease (LBD), Lou Gehrig's disease (ALS), Huntingdon's disease, and frontotemporal dementia (FTD).

The invention also relates to the use of a compound of formula I, or a pharmaceutically acceptable salt thereof, as defined above, in the manufacture of a medicament for the treatment or prevention of a disease in which modulation of the GPR3 receptor is beneficial, such as neurodegenerative diseases. Examples of such disease, disorder or condition are mentioned above.

The treatment and/or prevention of diseases in which modulation of modulation GPR3 receptor is beneficial and related pathology defined herein may be applied as a sole therapy or may involve, in addition to the compound of the invention, conjoint treatment with conventional therapy of value in treating one or more disease conditions referred to herein.

Such conjoint treatment and/or prevention of diseases may be achieved by way of simultaneous, sequential or separate dosing of the individual compounds of the invention and one or more additional therapeutic agents. Such combination products employ the compounds of the invention, or pharmaceutically acceptable salts, diastereomer, enantiomer, hydrates, solvates, or crystal or co-crystal thereof.

The invention relates to a pharmaceutical composition comprising (i) the compound of formula I or the list of compounds, or the pharmaceutical compositions as defined above, (ii) at least one additional therapeutic agent, or a pharmaceutically acceptable salt thereof, and (iii) a pharmaceutically acceptable excipient, carrier or diluent.

In another embodiment, the invention relates to a pharmaceutical composition comprising (i) the compound of formula I or the list of compounds, or the pharmaceutical compositions as defined above, and a pharmaceutically acceptable excipient, carrier or diluent, and (ii) at least one additional therapeutic agent, or a pharmaceutically acceptable salt thereof, and a pharmaceutically acceptable excipient, carrier or diluent.

In some aspects, at least one additional therapeutic agent is selected from the group comprising or consisting of cholesterase inhibitors (Donepezil, Galantamine, Rivastigmine), NMDA-receptor antagonists (Memantine), amyloid beta-directed monoclonal antibodies (Aducanumab), vitamin E, antipsychotics and/or anti-depressants.

### BRIEF DESCRIPTION OF THE DRAWING

The invention will now be explained more closely by the description of different embodiments of the invention and with reference to the appended figures.

FIG. 1 shows the activity of CBD, 6-OH-CBD, 7-OH-CBD and 7-COOH-CBD at the GPR3 receptor in the PathHunter™ β-arrestin2 recruitment assay.

FIG. 2 shows the activity of SR144528 and 7-COOH-CBD at the GPR3 receptor in the PathHunter™ β-arrestin2 recruitment assay.

FIG. 3 shows the efficacy of 7-COOH-CBD and compounds of formula I in a rat STZ-AD model.

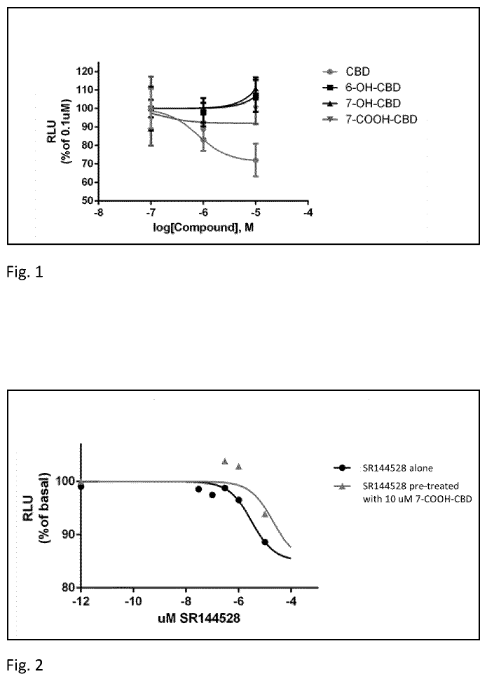

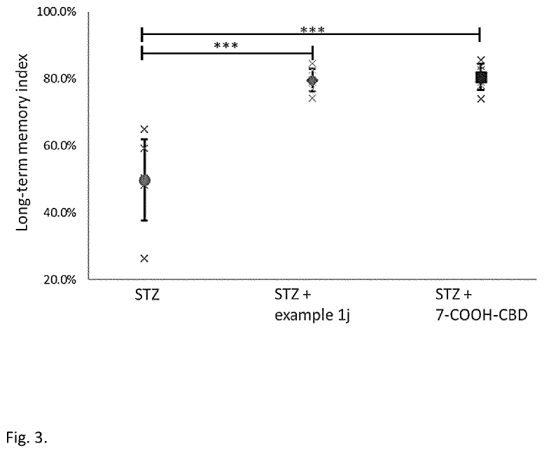

### DETAILED DESCRIPTION OF THE INVENTION

The definitions set forth in this application are intended to clarify terms used throughout this application. The term “herein” means the entire application.

As used herein, the term “disease” is intended to include disorder, condition or any equivalent is thereof.

As used herein, the term “patient” refers to a mammal, for example, a human.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

As used herein, a dash (“-”) that is not between two letters of symbols is used to indicate a point of attachment for a moiety of substituent. For example, —CN is a cyano group bonded through the carbon atom of said group.

As used herein, the term “compounds of the invention” refers to the compound of formula I, or any mixture thereof, or a pharmaceutically acceptable salt or crystals, co-crystal, hydrate or solvate, diastereomer or enantiomer thereof. Certain compounds may exist in multiple crystalline, co-crystalline, or amorphous forms.

As used herein, the term “optional” or “optionally” means that the subsequently described event or circumstance may but need not occur, and that the description includes instances where the event or circumstance occurs and instances where it does not.

As used herein, the terms “Cn”, used alone or as a suffix or prefix, is intended to include hydrocarbon-containing groups; n is an integer from 1 to 10.

As used herein, the term “halogen” or “halo”, used alone or as suffix or prefix, is intended to include bromine, chlorine, fluorine and iodine.

As used herein, the term “hetero”, used alone or as a suffix or prefix, is intended to include alkyl, cycloalkyl and aryl groups in which one or more of the carbon atoms (and certain associated hydrogen atoms) are independently replaced with the same or different hetero atoms (S, O or N) or heteroatomic groups. Examples of heteroatomic groups include, but are not limited to, —O—, —S—, —O—O—, —S—S—, —O—S—, NR, ═N—N═, —N═N— —N═N—NR— —PR— —P(O)₂— —POR—, —O—P(O)₂— —SO— —SO₂— —Sn(R)₂— and the like, where each R is independently selected from hydrogen, (substituted) alkyl, (substituted) aryl, (substituted) arylalkyl, (substituted) cycloalkyl, (substituted) heterocyclic alkyl, (substituted) heteroaryl, or (substituted) heteroarylalkyl.

Reference to, for example, a C₄₋₆heteroalkyl includes groups having five carbon atoms and one heteroatom, groups having four carbon atoms and two heteroatoms, etc. Reference to, for example, a C₆heterocycloalkyl includes aliphatic ring systems that have five carbon atoms and one heteroatom, aliphatic ring systems having four carbon atoms and two heteroatoms, etc. In certain embodiments, a heteroatomic group is selected from —O—, —S—, —NH—, —N(CH₃)—, and —SO₂—; and in certain embodiments, the heteroatomic group is —O—.

As used herein, the term “alkyl”, used alone or as a suffix or prefix, is intended to include both saturated or unsaturated, branched or straight chain, monovalent hydrocarbon radical derived by the removal of one hydrogen atom from a single carbon atom or atom or a parent alkane, alkene or alkyne. Examples include, but are not limited to methyl; ethyls, such as ethanyl, ethenyl, ethynyl; propyls such as propan-1-yl, propan-2-yl, prop-1-en-1-yl, prop-1-en-2-yl, prop-2-en-1-yl, prop-1-yn-1-yl, prop-2-yn-1-yl, etc; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yl-3-yl, but-3-yn-1-yl, etc.; and the like. The term “alkyl” is specifically intended to include groups having any degree or level of saturation, including groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl and alkynyl are used.

As used herein, the term “cycloalkyl”, used alone or as a suffix or prefix, is intended to include both saturated or partially unsaturated cyclic alkyl radical. Where a specific level of saturation is intended, the nomenclature cycloakanyl or cycloalkenyl is used. Examples of cycloalkyl groups include, but is not limited to, groups derived from cyclopropane, cyclobutene, cyclopentane, cyclohexane and the like. As used herein, the term “heterocycloalkyl” denotes a cycloalkyl comprising at least one heteroatom selected from O, N or S, such as morpholinyl, azetidinyl, azepane, piperidinyl, piperazinyl and the like. As used herein, the term “heterocycloalkenyl” denotes a partially unsaturated cyclic alkyl radical comprising at least one heteroatom selected from O, N or S, such as pyrrolinyl or pyranyl.

As used herein, the term “aryl” refers to either a monocyclic aromatic ring having 5 or 6 ring members or a multiple ring system having at least one carbocyclic aromatic ring fused to at least one carbocyclic aromatic ring, cycloalkyl ring or heterocycloalkyl ring. For example, aryl includes a phenyl ring fused to a 5- to 7-membered heterocycloalkyl ring containing one or more heteroatoms independently selected from N, O, and S.

As used herein, the term “heteroaryl” refers to a mono- or di-cyclic heteroaromatic ring having 5 or 13 ring members and wherein 1, 2, 3 or 4 ring atoms are independently selected from N, O and S. Examples include five-membered ring heteroaryls are thienyl, furyl, pyrrolyl, imidazolyl, thiazolyl, oxazolyl, pyrazolyl, pyrrolidinyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl or six-membered ring heteroaryls are pyridyl, pyrazinyl, pyrimidinyl, triazinyl and pyridazinyl.

As used herein, the term “leaving group” refers to an atom or group capable of being displaced by a nucleophile (e.g. halogen, alkoxycarbonyl, aryloxycarbonyl, mesyloxy, tosyloxy, trifluoromethanesulfnolyoxy, 2,4-dinitrophenoxy, methoxy, p-nitrophenlate, imidazolyl, and the like).

As used herein, the phrase “protecting group” means temporary substituents protecting a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been extensively reviewed (see, e.g. Jarowicki, K.; Kocienski, P. Perkin Trans. 1, 2001, issue 18, p. 2109).

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio and/or that are approved or approvable by a regulatory agency or body, for example the Food and Drug Administration or the European Medicines Agency.

As used herein, the term “salt” refers to forms of the disclosed compounds, wherein the parent compound is modified by making acid or base salts thereof, that possess the desired pharmacological activity of the parent compound. Generally, pharmaceutically acceptable salts of the compound of the invention as defined above may be obtained using standard procedures well known in the art, for example by reacting a sufficiently basic compound, for example an alkyl amine with a suitable acid, for example, hydrochloride or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aq. medium, followed by conventional purification techniques. Such salts include Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminium ion; or coordinates with an organic base such as ethanolamine, diethanolamine, triethanolamine, N-methylglucamine, and the like.

As used herein, the terms “crystal” or “crystalline solid” refer to a homogenous solid that is arranged in a definite and repeating three-dimensional pattern of atoms, ions and molecules, with smooth external surfaces with characteristic angles between them. Crystals, as opposed to amorphous solids, have a definite melting point.

As used herein, the term “co-crystal” refers to forms of the disclosed compounds, wherein the disclosed compound together with one or more different compounds (e.g. atoms, ions or molecules) forms a unique single phase, crystalline structure with unique physico-chemical properties as demonstrated by XRPD and/or melting points and/or solubility and/or chemical stability and/or mechanical properties.

As used herein, the term “compounds or pharmaceutically acceptable salts” include hydrates and solvates thereof.

A variety of compound of the invention as defined above may exist in particular geometric or stereoisomeric forms. The present invention takes into account all such compounds, including tautomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centres. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. All chiral, diastereomeric and racemic forms are intended, to be included in the scope of the invention, unless the specific stereochemistry or isomeric form is specifically indicated.

As used herein, “tautomer” means other structural isomers that exist in equilibrium resulting from the migration of a hydrogen atom. For example, keto-enol tautomerism occurs where the resulting compound has the properties of both a ketone and an unsaturated alcohol.

Compounds and salts/co-crystals described in this specification may be isotopically-labelled compounds (or “radio-labelled”). In that instance, one or more atoms are replaced by an atom having an atomic mass or mass number different from the atomic mass or mass number typically found in nature (i.e., naturally occurring). Examples of suitable isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁷⁵Br, ⁷⁶Br, ⁷⁷Br, ⁸²Br, ¹²³I, ¹²⁴I, ¹²⁵I, ¹³¹I. The radionuclide that is used will depend on the specific application of that radio-labelled derivative. For example, for in vitro receptor labelling and competition assays, compounds that incorporate ³H or ¹⁴C are often useful. For radio-imaging applications ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

The present invention is directed to a compound of formula I, a diastereomer, enantiomer, crystal or co-crystal thereof, hydrate, solvate, or a pharmaceutically acceptable salt, or mixture thereof,

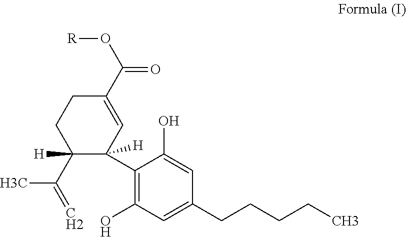

wherein R is selected from the group comprising or consisting of C₁₋₆alkyl, C₃₋₆alkenyl, C₁₋₆heteroalkyl, C₁₋₆heteroalkenyl, C₃₋₆cycloalkyl, C₃₋₆cycloalkenyl, C₃₋₆heterocycloalkyl, C₃₋₆heterocycloalkenyl, C₅₋₆aryl, C₅₋₆heteroraryl, C₃₋₆cycloalkylC₁₋₄alkyl, C₃₋₆cycloalkylC₃₋₄alkenyl, C₃-6heterocycloalkylC₁₋₄alkyl, C₃₋₆heterocycloalkylC₃₋₄alkenyl, C₅₋₆arylC₁₋₄alkyl, C₅₋₆arylC₃₋₄alkenyl, C₅-6heteroarylC₁₋₄alkyl, and C₅₋₆heteroarylC₃₋₄alkenyl which are optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl.

R may be C₁₋₆alkyl, which is optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR2, —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₁₋₆alkyl, which is optionally substituted with one or more R¹, wherein R¹ is independently selected from the group comprising or consisting of halogen, —OH, —CF₃, ═O, —NO₂, —C(O)NR²R³, —COOR², and —NR²R³, wherein R² and R³ are independently selected from the group comprising or consisting of hydrogen and C₁₋₆alkyl. Or R may be selected from the group comprising or consisting of methyl, ethyl, propyl, butyl, pentyl, hexyl, cyanomethyl, methoxymethyl, 2-oxoethyl, 2-aminoethyl, 2-cyanoethyl, 1-methylethyl, 2-methylethyl, 1,1-dimethylethyl, 2-cyano-1-methylethyl, 2,2-difluoroethyl, 2,2,2-trifluoroethyl, 2-(dimethylamino)ethyl, 2-amino-2-ethoxyethyl, 1-methylpropyl, 2-methylpropyl, 3-aminopropyl, 1,1-dimethylpropyl, 2,2-dimethylpropyl, 2-cyanopropyl, 3-(ethenyloxy)propyl, 3-oxobutyl, 1-methylbutyl, 2-methylbutyl, 3-methylbutyl, 1,1-dimethylbutyl and 2-amino-1-methylethyl, or the group comprising or consisting of methyl, ethyl, propyl, butyl, pentyl hexyl, 1-methylethyl, 2-methylethyl, 1,1-dimethylethyl, 1-methylpropyl, 2-methylpropyl, 1,1-dimethylpropyl, 2,2-dimethylpropyl, 1-methylbutyl, 2-methylbutyl, 3-methylbutyl and 1,1-dimethylbutyl. Or R may be selected from the group comprising or consisting of methyl, ethyl, propyl and butyl.

R may be C₃₋₆alkenyl, which is optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₃₋₆alkenyl, which is optionally substituted with one or more R¹, wherein R¹ is independently selected from the group comprising or consisting of halogen, —OH, —CF₃, ═O, —NO₂, —C(O)NR²R³, —COOR², and —N R²R³, wherein R² and R³ are independently selected from the group comprising or consisting of hydrogen and C₁₋₆alkyl. Or R may be selected from the group comprising or consisting of propenyl, 2-propen-1-yl, 2-methyl-2-propen-1-yl, butenyl and 3-buten-1-yl. Or R may be 3-buten-1-yl.

R may be C₃₋₆cycloalkyl or C₃₋₆cycloalkylC₁₋₄alkyl, which are optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —N R⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₃₋₆cycloalkyl or C₃₋₆cycloalkylC₁₋₄alkyl, which are optionally substituted with one or more R¹, wherein R¹ is independently selected from the group comprising or consisting of halogen, —OH, —CF₃, ═O, —NO₂, —C(O)NR²R³, —COOR², and —NR²R³, wherein R² and R³ are independently selected from the group comprising or consisting of hydrogen and C₁₋₆alkyl. Or R may be selected from the group comprising or consisting of cyclopropyl, 1-(hydroxymethyl)cyclopropyl, cyclobutyl, 3-hydroxy-cyclobutyl, 3-methoxy-cyclobutyl, 1-cyanocyclobutyl, cyclopentyl, 1-methylcyclopentyl, 3-methylcyclopentyl, 2-hydroxycyclopentyl, 3-hydroxycyclopentyl, 2,3-dihydroxycyclopentyl, cyclohexyl, cyclopropylmethyl, cyclobutylmethyl, cyclopentylmethyl, (1-hydroxycyclobutyl)methyl, (1-hydroxycyclobutyl)methyl, (3-hydroxycyclobutyl)methyl, (3-methoxycyclobutyl)-methyl, 2-cyclopropylethyl, 1-cyclobutylethyl and 2-cyclobutylethyl. Or R may be selected from the group comprising or consisting of cyclopropyl, cyclobutyl and cyclopentyl.

R may be C₃₋₆cycloalkenyl, which is optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₃₋₆cycloalkenyl, which is optionally substituted with one or more R¹, wherein R¹ is independently selected from the group comprising or consisting of halogen, —OH, —CF₃, ═O, —NO₂, —C(O)NR²R³, —COOR², and —NR²R³, wherein R² and R³ are independently selected from the group comprising or consisting of hydrogen and C₁₋₆alkyl. Or R may be selected from the group comprising or consisting of cyclopentenyl, 1-cyclohexen-1-yl, 1-cyclopenten-1-yl, amino-cyclopentenyl and fluoro-cyclopentenyl.

R may be C₃₋₆heterocycloalkyl, C₃₋₆heterocycloalkenyl C₃₋₆heterocycloalkylC₁₋₄alkyl or C₃₋₆heterocycloalkenylC₁₋₄alkyl, which are optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR5, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₃₋₆heterocycloalkyl, C₃₋₆heterocycloalkenyl C₃₋₆heterocycloalkylC₁₋₄ alkyl or C₃₋₆heterocycloalkenylC₁₋₄alkyl, which are optionally substituted with one or more R¹, wherein R¹ is independently selected from the group comprising or consisting of halogen, —OH, —CF₃, ═O, —NO₂, —C(O)NR²R³, —COOR², —R⁵, —OR⁵ and —NR²R³, wherein R² and R³ are independently selected from the group comprising or consisting of hydrogen and C₁₋₆alkyl. Or R may be selected from the group comprising or consisting of 4-piperidinyl, 1-methyl-3-pyrrolidinyl, piperazinyl and 4-methyl-1-piperazinyl)methyl. Or R may be selected from the group comprising or consisting of piperidinomethyl, 1-piperazinylmethyl, (4-methyl-1-piperazinyl)methyl, and (5-oxo-3-pyrrolin-2yl)methyl.

R may be C₅₋₆aryl, C₅₋₆heteroraryl, C₅₋₆arylC₁₋₄alkyl or C₅₋₆heterorarylC₁₋₄alkyl, which are optionally substituted with one or more R¹, wherein R¹ is independently selected from the group comprising or consisting of halogen, —OH, —SH, —CN, —CF₃, ═O, —NO₂, —C(O)NR²R³, —R², —OR², —SR², —C(O)R², —COOR² and —NR²R³, wherein R² and R³ are independently selected from the group comprising or consisting of hydrogen, C₁₋₆alkyl, C₃₋₆alkenyl and C₃₋₆cycloalkyl, and optionally substituted with one or more R⁴, wherein R⁴ is selected from the group comprising or consisting of halogen, —OH, —SH, —CN, —CF₃, ═O, —NO₂, —C(O)NR⁵R⁶, —R⁵, —OR⁵, —SR⁵, —C(O)R⁵, —COOR⁵ and —NR⁵R⁶, wherein R⁵ and R⁶ are independently selected from the group comprising or consisting of hydrogen, C₁₋₆alkyl, C₃₋₆alkenyl and C₃₋₆cycloalkyl. Or R may be C₅₋₆aryl, C₅₋₆heteroraryl, C₅₋₆arylC₁₋₄alkyl or C₅₋₆heterorarylC₁₋₄alkyl, which are optionally substituted with one or more R¹, wherein R¹ is independently selected from the group comprising or consisting of halogen, —OH, —CF₃, ═O, —NO₂, —C(O)NR²R³, —COOR², and —NR²R³, wherein R² and R³ are independently selected from the group comprising or consisting of hydrogen and C₁₋₆alkyl. Or R may be selected from the group comprising or consisting of 1H-pyrrol-2-yl, 1H-pyrrol-3-yl, tetrahydro-3-furanyl, 3-pyridinyl, 2-thienyl, 1H-pyrrol-2-ylmethyl, 1H-pyrrol-3-ylmethyl, 2-furanylmethyl, 1H-imidazol-2-yl, 1-methyl-1H-pyrazol-3-yl, 5-methyl-2-furanyl, 4-methyl-4H-1,2,4-triazol-3-yl, 1H-1,2,4-triazol-5-ylmethyl, 2-thiazolyl, 1,3,4-thiadiazol-2-yl, phenyl, (tetrahydro-2-furanyl)methyl and (tetrahydro-3-furanyl)methyl. Or R may be (p-chlorophenyl)methyl or (o-methoxyphenyl)methyl.

The invention also relates to any one of compound or a pharmaceutically acceptable salt, crystal or co-crystal thereof, selected from the group comprising or consisting of
(3R-trans)-3-(2,6-dihydroxy-4-pentylphenyl)-4-(1-methylethenyl)-1-cyclohexene-1-carboxylic acid methyl ester,
(3R-trans)-3-(2,6-dihydroxy-4-pentylphenyl)-4-(1-methylethenyl)-1-cyclohexene-1-carboxylic acid ethyl ester,
(3R-trans)-3-(2,6-dihydroxy-4-pentylphenyl)-4-(1-methylethenyl)-1-cyclohexene-1-carboxylic acid propyl ester,
(3R-trans)-3-(2,6-dihydroxy-4-pentylphenyl)-4-(1-methylethenyl)-1-cyclohexene-1-carboxylic acid isopropyl ester,
(3R-trans)-3-(2,6-dihydroxy-4-pentylphenyl)-4-(1-methylethenyl)-1-cyclohexene-1-carboxylic acid n-butyl ester,
(3R-trans)-3-(2,6-dihydroxy-4-pentylphenyl)-4-(1-methylethenyl)-1-cyclohexene-1-carboxylic acid 3-butenyl ester,
(3R-trans)-3-(2,6-dihydroxy-4-pentylphenyl)-4-(1-methylethenyl)-1-cyclohexene-1-carboxylic acid 1-methylpropyl ester,
(3R-trans)-3-(2,6-dihydroxy-4-pentylphenyl)-4-(1-methylethenyl)-1-cyclohexene-1-carboxylic acid isobutyl ester, and
(3R-trans)-3-(2,6-dihydroxy-4-pentylphenyl)-4-(1-methylethenyl)-1-cyclohexene-1-carboxylic acid tertbutyl ester.

The invention also relates to any one of compound or a pharmaceutically acceptable salt, crystal or co-crystal thereof, selected from the group comprising or consisting of
(3R-trans)-3-(2,6-dihydroxy-4-pentylphenyl)-4-(1-methylethenyl)-1-cyclohexene-1-carboxylic acid cyclopropane ester,
(3R-trans)-3-(2,6-dihydroxy-4-pentylphenyl)-4-(1-methylethenyl)-1-cyclohexene-1-carboxylic acid cyclobutane ester,
(3R-trans)-3-(2,6-dihydroxy-4-pentylphenyl)-4-(1-methylethenyl)-1-cyclohexene-1-carboxylic acid cyclopentane ester,
(3R-trans)-3-(2,6-dihydroxy-4-pentylphenyl)-4-(1-methylethenyl)-1-cyclohexene-1-carboxylic acid 1-cyclopentene ester,
(3R-trans)-3-(2,6-dihydroxy-4-pentylphenyl)-4-(1-methylethenyl)-1-cyclohexene-1-carboxylic acid 3-cyclopentene ester,
(3R-trans)-3-(2,6-dihydroxy-4-pentylphenyl)-4-(1-methylethenyl)-1-cyclohexene-1-carboxylic acid 2-amino-1-cyclopentene ester, and
(3R-trans)-3-(2,6-dihydroxy-4-pentylphenyl)-4-(1-methylethenyl)-1-cyclohexene-1-carboxylic acid 2-fluoro-1-cyclopentene ester.

The invention also relates to any one of compound or a pharmaceutically acceptable salt, crystal or co-crystal thereof, selected from the group comprising or consisting of
(3R-trans)-3-(2,6-dihydroxy-4-pentylphenyl)-4-(1-methylethenyl)-1-cyclohexene-1-carboxylic acid piperidinomethyl ester,
(3R-trans)-3-(2,6-dihydroxy-4-pentylphenyl)-4-(1-methylethenyl)-1-cyclohexene-1-carboxylic acid 1-piperazinylmethyl ester,
(3R-trans)-3-(2,6-dihydroxy-4-pentylphenyl)-4-(1-methylethenyl)-1-cyclohexene-1-carboxylic acid (4-methyl-1-piperazinyl)methyl ester,
(3R-trans)-3-(2,6-dihydroxy-4-pentylphenyl)-4-(1-methylethenyl)-1-cyclohexene-1-carboxylic acid (p-chlorophenyl)methyl ester,
(3R-trans)-3-(2,6-dihydroxy-4-pentylphenyl)-4-(1-methylethenyl)-1-cyclohexene-1-carboxylic acid (o-methoxyphenyl)methyl ester, and
(3R-trans)-3-(2,6-dihydroxy-4-pentylphenyl)-4-(1-methylethenyl)-1-cyclohexene-1-carboxylic acid (5-oxo-3pyrrolin-2yl)methyl ester or a diastereomer, enantiomer, hydrates, solvates, or a pharmaceutically acceptable salt, or mixture thereof.

These compounds fall within the scope of compounds of formula (I). It is to be understood that this list of compounds is included in the wording “compound of formula I, or a pharmaceutically acceptable salt, crystals or co-crystals, hydrates or solvents, diastereomer, enantiomer thereof” or “compound(s) of the invention”, as used in embodiments related to uses, pharmaceutical compositions or processes, unless specified otherwise.

### Medical Use

The compounds of the present invention are believed to be useful as a medicament in therapy. The compounds are especially believed to be useful in the prevention, progression prophylaxis and/or treatment of neurodegenerative diseases and in the diseases selected from the group comprising or containing Alzheimer disease (AD), Parkinson's disease (PD), Lewy Body disease (LBD), Lou Gehrig's disease (ALS), Huntingdon's disease, and frontotemporal dementia (FTD).

The compound of formula I, as defined above, for use in the prevention, progression prophylaxis and/or treatment of autoimmune diseases, whereby the ratio of pharmacokinetic parameters (Css, Cmax and/or AUC) of 7-COOH-CBD to the compound of formula I in blood plasma after administration of the compound as defined above, is larger than 1.000.000, or 100.000, or 10.000 or 1000 or 100 or 10 or 1.

In one embodiment of the invention, the ratio of pharmacokinetic parameters (Css, Cmax and/or AUC) of 7-COOH-CBD to the compound of formula I in blood plasma after administration of the compound as defined above, is larger than 1.000.000, or 100.000, or 10.000.

In another embodiment of the invention, the levels of the compound of formula I in blood plasma after administration are below the level of detection. The compound of formula I are expected to be hydrolysed immediately by esterase upon entry into the blood. Therefore, the concentration of the esters of formula I in blood is expected to be below the level of detection.

The route of administration of compounds of the present invention may be orally, parenteral, buccal, vaginal, rectal, inhalation, insufflation, sublingually, intramuscularly, subcutaneously, topically, intranasally, intraperitoneally, intrathoracically, intravenously, epidurally, intrathecally, and intracerebroventricularly. The route of administration of compounds of the present invention may be orally.

The optimum dosage and frequency of administration will depend on the particular condition being treated and its severity; the age, sex, size and weight, and general physical condition of the particular patient; other medication the patient may be taking; the route of administration; the formulation; and various other factors known to physicians and others skilled in the art. For example, the frequency of administration will vary for the disease being treated from 1 to 4 times daily. The quantity of the compounds of the invention to be administered will vary for the patient being treated and will vary from about 100 ng/kg of body weight to 100 mg/kg of body weight per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods and uses of the invention.

The compounds of the invention may be used on their own but will generally be administered in the form of a pharmaceutical composition in which the compound of the invention (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

For preparing pharmaceutical compositions from the compounds of the invention, inert, pharmaceutically acceptable carriers can be either solid or liquid. Solid form compositions include powders, tablets, dispersible granules, capsules, cachets, and suppositories. Liquid forms include parenteral forms for either intravenous or sub cutaneous or intra-muscular administration, or syrups, and liquid capsules for oral administration.

A solid carrier can be one or more substances, which may also act as diluents, flavouring agents, solubilizers, lubricants, suspending agents, binders, or tablet disintegrating agents; it can also be an encapsulating material.

Depending on the mode of administration, the pharmaceutical composition will preferably comprise from 0.05 to 99% wt (percent by weight), more preferably from 0.05 to 90% wt, still more preferably from 0.10 to 70% wt, and even more preferably from 0.10 to 50% wt, of active ingredient, all percentages by weight being based on total composition.

Compounds of the present invention may be used in conjoint therapy with other conventional therapies used to linger symptoms accompanying the progressive physical disability and mental deterioration caused by the degenerative disease. For example, patients with AD will generally receive cholesterase inhibitors (Donepezil, Galantamine, Rivastigmine), and/or an NMDA-receptor antagonist (Memantine), and/or an amyloid beta-directed monoclonal antibody (Aducanumab), and/or vitamin E, and/or antipsychotics, and/or anti-depressants to treat anxiety or depression associated with the disease.

### Preparation of Compounds.

Compounds of the present invention can be prepared by the processes described below. Throughout the following description of such processes, it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999.

### General Methods

All solvents used were of analytical grade and commercially available anhydrous solvents were routinely used for reactions. All solvents used were dried prior to use, for example by a passage is over a column of activated alumina or by distillation over calcium hydride. All glassware used was oven-dried and sealed with rubber septa under a nitrogen atmosphere. Starting materials used were available from commercial sources or prepared according to literature procedures. Room temperature refers to temperatures ranging from 16 to 25° C. Solvent mixture compositions are given as volume percentages or volume ratios. The produced examples were characterized regarding identity, crystal modification, thermal properties, melting point, solubility in water, purity, and hygroscopicity using the following methods:

Thin layer chromatography (TLC) was performed on Merck TLC-plates (Silica gel 60 F254) and spots were UV visualized. Straight phase flash column chromatography (“flash chromatography”) was manually performed on Merck Silica gel 60 (0.040-0.063 mm).

LC/MS/MS analysis equipment: API 4000 equipped with an Agilent 1100 HPLC and a Leap Technologies auto-sampler. A HPLC Phenomenex Onyx Monolithic C18 (CHO-7644) column at a temperature of 35° C., flow rate of 2.0 ml/min, injection volume of 30 μL, and a 3 min run time was used. Mobile phase AI was 01% formic acid in water and Mobile phase AII was 0.1% formic acid in ACN. The gradient was 2% All in AI at t=0 to t=0.13 min, 5% AII in AI at t=1.4 to 2.2 min and 98% AII in AI at t=2.3 to 3.0 min. The ratio was determined using a negative ion mode (QI 128.94; Q2 71). Abbreviations
ACN acetonitrile
Aq. Aqueous
DCM dichloromethane
DMF N,N-dimethyl formamide
DMSO dimethyl sulfoxide
EtOAc ethyl acetate EtOH ethanol
eq. or equiv. equivalent
h hour(s)
HPLC high performance liquid chromatography
LCMS liquid chromatography mass spectrometry
MeOH methanol
min minute(s)
MS mass spectrometry
NMR nuclear magnetic resonance
r.t. room temperature, i.e. between 16 to 25*C
sat. saturated
THF tetrahydrofuran
TLC thin layer chromatography

### Naming Compounds:

Compounds have been named using CambridgeSoft MedChem ELN v2.2 or ACD/Name, version 10.0, or 10.06, or version 12.01, software from Advanced Chemistry Development, Inc. (ACD/Labs), Toronto ON, Canada, www.acdlabs.com, or Lexichem, version 1.9, software from OpenEye. General synthetic methods, or ChemDoodle-D2 IUPAC naming tool.

General synthetic methods useful in the synthesis of compounds described herein are available in the art. Starting materials useful for preparing compounds and intermediates thereof and/or practicing methods described herein are commercially available or can be prepared by well-known synthetic methods. The methods presented in the schemes and examples provided by the present disclosure are illustrative rather than comprehensive. It will be apparent to those skilled in the art that many modifications, both to materials and methods, may be practiced without departing from the scope of the disclosure.

For example, a compound of formula I may be obtained via the synthetic methods illustrated in the following steps and as described by Heller and Sarpong in 2010 and 2011 for selective esterification of the COOH group without a need to protect the OH groups from alkylation:

### Preparation of the Imidazole Carbamate Reagent R-ImC

### Procedure A:

Imidazole (3.75 g, 55 mmol) was dissolved in dry THF (50 ml) and the resulting solution was stirred with cooling to 0° C. Allyl chloroformate (2.98 ml, 28 mmol) was added dropwise and the resulting white suspension was vigorously stirred at 0° C. for 1 h and then at room temperature for 4 h. The mixture was filtered, and the filter cake was washed with ether (50 ml). The filtrate was concentrated in vacuo and the colourless concentrate obtained was dissolved in diethyl ether (100 ml), washed with water (2×50 ml), dried over MgSO₄, and concentrated in vacuo to afford the desired allyl 1-imidazolecarboxylate as a colourless oil.

### Procedure B

1,1′-Carbonyldiimidazole (1.14 g, 9.00 mmol) was dissolved in DCM (20 ml) and the resulting solution was stirred with cooling to 0° C. (±)-3-buten-2-ol (0.520 ml, 6.00 mmol) was then added dropwise. The reaction mixture was stirred at 0° C. for 1 h, and then at room temperature for 16 h. The homogeneous mixture was then diluted with DCM (20 ml), washed with water (2×20 ml), dried over MgSO₄, and concentrated in vacuo to afford the desired (±)-but-3-en-2-yl 1-imidazolecarboxylate a colourless oil (0.877 g, 90%).

### Preparation of a Compound of Formula (I)

The general procedure for the preparation of compounds of the invention (Formula I) is as follows: 7-COOH-CBD (0.5 mmol) and the imidazole carbamate reagent R-ImC (1.0 mmol), which has been obtained either commercially or by using Procedure A or Procedure B, are placed in a reaction vessel together with a magnetic stir bar. The reaction is carried out under nitrogen and kept dry. Acetonitrile (1 mL) is added and the reaction mixture is stirred at room temperature for 15 min, before being heated (60-80° C.) overnight (18-28 h). The mixture is cooled to room temperature and the solvent evaporated in vacuo. The resulting residue is dissolved diethylether (25 mL) and washed with 1 M HCl (10 mL). The aqueous layer is back extracted with diethylether (25 mL) and the organic fractions are combined washed with a saturated solution of NaHCO₃ and with a saturated solution of NaCl, dried over MgSO₄ and concentrated in vacuo to obtain the desired ester. The ester is further purified using either flash chromatography over silica and/or recrystallization from a suitable organic solvent mixture.

The process of preparation of a compound of formula I, as defined above substantially pure compounds are provided, or compounds having a purity of a least 80% by weight, or at least 85% by weight, or at least 90% by weight, or at least 90, 91, 92, 93, 94, 95, 96, 97, 98, 99 or 100% by weight.

### Experimental Part

### Example 1

Below follow several illustrative, non-limiting examples of the compounds of the invention.

### (3R-trans)-3-(2,6-dihydroxy-4-pentylphenyl)-4-(1-methylethenyl)-1-cyclohexene-1-carboxylic acid methyl ester (Example 1a)

7-COOH-CBD (100 mg; 0.5 mmol) and methyl 1-imidazolecarboxylate (1.0 mmol) are placed in a reaction vessel under nitrogen together with a magnetic stir bar and acetonitrile (1 mL) is added. The reaction mixture is stirred at room temperature for 15 min, before being heated to 80° C. for 24 h. The mixture is cooled to room temperature and the solvent evaporated in vacuo.

The resulting residue is dissolved in diethylether (25 mL) and washed with 1 M HCl. The aqueous layer is back extracted with diethylether and the combined organic fractions are first washed with a saturated solution of NaHCO₃ and then with a saturated solution of NaCl. The organic solvent is dried over MgSO₄ and concentrated in vacuo, yielding 80% of the title compound as an off-white solid. MS (ESI): m/z 359.44 (M+H).
(3R-trans)-3-(2,6-dihydroxy-4-pentylphenyl)-4-(1-methylethenyl)-1-cyclohexene-1-carboxylic acid ethyl ester (1b) MS (ESI): m/z 373.44 (M+H), (example 1b).
(3R-trans)-3-(2,6-dihydroxy-4-pentylphenyl)-4-(1-methylethenyl)-1-cyclohexene-1-carboxylic acid propyl ester (1c) MS (ESI): m/z 387.44 (M+H), (example c).
(3R-trans)-3-(2,6-dihydroxy-4-pentylphenyl)-4-(1-methylethenyl)-1-cyclohexene-1-carboxylic acid isopropyl ester (1d) MS (ESI): m/z 387.44 (M+H), (example 1d).
(3R-trans)-3-(2,6-dihydroxy-4-pentylphenyl)-4-(1-methylethenyl)-1-cyclohexene-1-carboxylic acid n-butyl ester (1e) MS (ESI): m/z 401.44 (M+H), (example 1e).
(3R-trans)-3-(2,6-dihydroxy-4-pentylphenyl)-4-(1-methylethenyl)-1-cyclohexene-1-carboxylic acid 3-butenyl ester (1f) MS (ESI): m/z 399.44 (M+H), (example 1f).
(3R-trans)-3-(2,6-dihydroxy-4-pentylphenyl)-4-(1-methylethenyl)-1-cyclohexene-1-carboxylic acid 1-methylpropyl ester (1g) MS (ESI): m/z 401.44 (M+H), (example 1g).
(3R-trans)-3-(2,6-dihydroxy-4-pentylphenyl)-4-(1-methylethenyl)-1-cyclohexene-1-carboxylic acid isobutyl ester (1h) MS (ESI): m/z 401.44 (M+H), (example 1h).
(3R-trans)-3-(2,6-dihydroxy-4-pentylphenyl)-4-(1-methylethenyl)-1-cyclohexene-1-carboxylic acid tertbutyl ester (1i) MS (ESI): m/z 401.44 (M+H), (example 1i).
(3R-trans)-3-(2,6-dihydroxy-4-pentylphenyl)-4-(1-methylethenyl)-1-cyclohexene-1-carboxylic acid cyclopropane ester (1j) MS (ESI): m/z 384.44 (M+H), (example 1j).
(3R-trans)-3-(2,6-dihydroxy-4-pentylphenyl)-4-(1-methylethenyl)-1-cyclohexene-1-carboxylic acid cyclobutane ester (1k) MS (ESI): m/z 398.44 (M+H), (example 1k).
(3R-trans)-3-(2,6-dihydroxy-4-pentylphenyl)-4-(1-methylethenyl)-1-cyclohexene-1-carboxylic acid cyclopentane ester (1l) MS (ESI): m/z 412.44 (M+H), (example 1l).
(3R-trans)-3-(2,6-dihydroxy-4-pentylphenyl)-4-(1-methylethenyl)-1-cyclohexene-1-carboxylic acid 1-cyclopentene ester (1m) MS (ESI): m/z 411.44 (M+H), (example 1m).
(3R-trans)-3-(2,6-dihydroxy-4-pentylphenyl)-4-(1-methylethenyl)-1-cyclohexene-1-carboxylic acid 3-cyclopentene ester (1n) MS (ESI): m/z 411.44 (M+H), (example 1n).
(3R-trans)-3-(2,6-dihydroxy-4-pentylphenyl)-4-(1-methylethenyl)-1-cyclohexene-1-carboxylic acid 2-amino-1-cyclopentene ester (1o) MS (ESI): m/z 426.44 (M+H), (example 1o).
(3R-trans)-3-(2,6-dihydroxy-4-pentylphenyl)-4-(1-methylethenyl)-1-cyclohexene-1-carboxylic acid 2-fluoro-1-cyclopentene ester (1p) MS (ESI): m/z 429.43 (M+H), (example 1p).
(3R-trans)-3-(2,6-dihydroxy-4-pentylphenyl)-4-(1-methylethenyl)-1-cyclohexene-1-carboxylic acid piperidinomethyl ester (1q) MS (ESI): m/z 442.44 (M+H), (example 1q).
(3R-trans)-3-(2,6-dihydroxy-4-pentylphenyl)-4-(1-methylethenyl)-1-cyclohexene-1-carboxylic acid 1-piperazinylmethyl ester (1r) MS (ESI): m/z 443.44 (M+H), (example 1r).
(3R-trans)-3-(2,6-dihydroxy-4-pentylphenyl)-4-(1-methylethenyl)-1-cyclohexene-1-carboxylic acid (4-methyl-1-piperazinyl)methyl ester (1s) MS (ESI): m/z 457.44 (M+H), (example 1s).
(3R-trans)-3-(2,6-dihydroxy-4-pentylphenyl)-4-(1-methylethenyl)-1-cyclohexene-1-carboxylic acid (p-chlorophenyl)methyl ester (1t) MS (ESI): m/z 457.45 (M+H), (example 1t).
(3R-trans)-3-(2,6-dihydroxy-4-pentylphenyl)-4-(1-methylethenyl)-1-cyclohexene-1-carboxylic acid (o-methoxyphenyl)methyl ester (1u) MS (ESI): m/z 453.44 (M+H), (example 1u).
(3R-trans)-3-(2,6-dihydroxy-4-pentylphenyl)-4-(1-methylethenyl)-1-cyclohexene-1-carboxylic acid (5-oxo-3pyrrolin-2yl)methyl ester (1v) MS (ESI): m/z 440.44 (M+H), (example 1v).

For a prodrug, it is desirable that it is transformed into the active drug after being administered parenterally or taken up from the gastrointestinal lumen, e.g. in either the enterocytes lining of the gastrointestinal lumen or in the blood. A useful level of stability can at least in part be determined by the mechanism of transformation and pharmacokinetics of the prodrug. In general, prodrugs that are labile in intestinal S9 fraction, and/or plasma, and/or liver S9 fraction can be useful as a prodrug. The results of tests, such as those described in example 2, for determining the enzymatic cleavage of prodrugs in vitro can be used to select prodrugs for in vivo testing. The stabilities of prodrugs can be evaluated in one or more in vitro systems using a variety of preparations following methods known in the art. For example, methods used to determine the stability of prodrugs in plasma, liver S9 and intestine S9 preparations, or colonic wash assays have been extensively described in the literature and are offered by commercial vendors.

### Example 2

### Methods for Determining the Biological Stability of Compounds 1a to 1u In Vitro.

### Preparation of Lysates and Study Protocol.

Preparation of 400 μM test compound in 0.1 M Tris buffer, pH 7.4:10 μl of a 20 mM aq. stock solution is added to 490 ml of 0.1 M Tris buffer, pH 7.4 and kept at −20° C. until the time of use.

Enzymatic stability assay: 90 μl per time point of lysate (rat plasma, human plasma) is aliquoted to designated tubes on a cluster plate (t=0s, 60 s, 5 min, 20 min and 60 min) and preincubated for 10 min at 37° C. For the t=0 s, 90 μl of lysate is added to 200 μl of ice cold 100% ethanol, after which the sample is thoroughly mixed. To this mixture, 10 μl of a 10 μM test compound solution in TRIS buffer, pH 7.4 is added. The sample is mixed again and kept at −20° C. until the time of analysis. For the other time points, 10 μM test compound solutions in TRIS buffer, pH 7.4 is added and 100 μl of the resulting 1 μM lysate-test mixture that is kept at 37° C. is withdrawn at the appropriate time points and added to 200 μl of ice cold 100% ethanol. The mixtures are thoroughly mixed and kept at −20° C. until the time of analysis. For analysis, the samples are defrosted and centrifuged for 10 min at 6000 rpm at 4° C., after which the supernatant is transferred to individual vials suitable for use in the LC-MS/MS equipment. Samples are analyzed with LC-MS/MS to determine the parent prodrug cleavage.

Results: compounds 1a-1u are converted to 7-COOH-CBD in rat and human plasma within at least 60 min.

The neurodegenerative Alzheimer disease is characterized by the disposition of neurotoxic beta amyloid plaques and Tau-tangles. The production of these are stimulated by the constitutively active GPR3 receptor. CBD acts as an inverse agonist at the GPR3 receptor, inducing an altered pharmacological response from the natural effects of the GPR3 receptor's activity leading to clinically unknown response.

### Example 3

The effects of ligands on the activity of the GPR3 receptor can be demonstrated with the use of the in vitro PathHunter™ β-arrestin2 recruitment assay.

### Materials:

The PathHunter™ eXpress kits containing Chinese hamster ovary (CHO)-K1 cells co-expressing EA-O-arrestin2 human and GPR3-PK were purchased from DiscoverX (Fremont, CA). All cannabinoid ligands were purchased from BDG Synthesis (Wellington, New Zealand).

### Pathhunter™ β-Arrestin2 Recruitment Assays:

PathHunter™ eXpress kits were used to measure GPR3-mediated β-arrestin2 recruitment following manufacturer's instructions. In this system, Chinese hamster ovary (CHO)-K1 cell line are stably expressing the GPR3 receptors which were fused to a “ProLink (PK)” fragment of βi-galactosidase to form GPR3-PK. The remaining sequence of β-galactosidase enzyme acceptor (EA) was linked to β-arrestin2 to form EA-β-arrestin2. Recruitment of Si-arrestin2 by receptor activation causes complementation of the two enzyme fragments. Levels of the active enzyme are the direct result of β-arrestin2 recruitment caused by receptor activation and quantified using the PathHunter detection reagent containing β-galactosidase substrates. Cells were plated in DiscoverX cell plating reagent1 in 384-well plates and cultured for 24-48 hours prior to experimentation in a humidified atmosphere at 37° C. and 5% CO2. Cannabinoid ligands were diluted in DiscoverX cell plating reagent1. Cells were then incubated with ligand at 37° C. following manufacturer's recommendations, followed by incubation with detection reagent in the dark for 1 hour at 23° C. Luminescence signal was then detected using a TECAN GENios Pro microplate reader.

### Data Analysis:

Ligand-induced changes in R-arrestin2 recruitment to GPR3 were expressed as percent basal relative luminescence units, which was calculated by dividing luminescence readings in the presence of ligands by basal luminescence readings, times 100. Concentration-response curves were generated by performing nonlinear regression analysis using GraphPad Prism (GraphPad Software, La Jolla, CA). Data were analyzed using one-way analysis of variance (ANOVA) followed by NewmanKeuls post-test. Data points shown are presented as mean±SEM, and were obtained from three independent experiments performed in quadruplicate.

### Study 1:

To determine whether CBD and its human metabolites 6-OH-CBD, 7-OH-CBD and 7-COOH-CBD are capable of changing S3-arrestin2 recruitment to GPR3, β-arrestin2 recruitment assays were performed using 0.1 μM and 1 μM and 10 μM concentrations.

### Study 2:

To demonstrate that 7-COOH-CBD binds to the GPR3 receptor, a competition experiment is performed where cells are incubated with 10 μM 7-COOH-CBD to which ascending concentrations of SR144528, a test compound that is structurally different from the tested cannabinoids and that acts as a GPR3 inverse agonist with an IC50 of 16 μM, is added.

### Results:

As expected, CBD itself acts as an inverse agonist in this assay. Both 6-OH-CBD and 7-OH-CBD act as weak agonists at the GPR3 receptor. Only the 7-COOH-CBD metabolite acts as a full antagonist at the GPR3 receptor, effectively shutting off the effects mediated by this receptor.

In the competition experiment between the structurally different SR144528 and 7-COOH-CBD, the latter indeed displaces the inverse agonist from the GPR3 binding site, shifting the IC50 of SR144528 with one log unit to 0.2 mM.

Compounds of formula I, which are enzymatically converted to 7-COOH-CBD will exclusively silence the GPR3 receptor, which clinically results in the prevention, prophylaxis of progression, and/or treatment of a disease in which the modulation of the G-protein coupled receptor 3 (GPR3) and/or the amount of Tau protein in a cell and/or the amount of neurofibrillary tangles and/or the amount of the peptide fragment amyloid beta and/or the amount of beta amyloid plaques in the brain and/or in cerebral blood vessels is beneficial.

### Example 4

The effects of ligands on the memory can be demonstrated with the use of the in vivo intracerebroventricular (icv) injection of streptozotocin (stz) rat model of Alzheimer Disease.

This in vivo model represents a model for the most frequent form of Alzheimer Disease, the late onset or ‘sporadic’ form. The icv injection of stz in the rat brain induces pathological changes that are observed in patients with Alzheimer Disease, like accumulation of beta-amyloid and Tau-protein and the well-known cognitive impairment. This well-known model was first described in the 90-ies by Mayer et al. (1990) and Lannert et al. (1998). The procedure for the creation of the model has recently been described in detail by Moreiro-Silva et al. (2019). In short, a 1-3 mg/kg dose of stz is injected bilateraly by icv injection in Wistar rats, and the animals are allowed to recover for a period of 7 days, after which animals can be evaluated for neurodegenerative changes in behaviour and/or by imaging of the brain.

### Data Analysis:

The novel object recognition data were analyzed using the Student's t-test, with differences being considered significant if p<0.05.

### Study:

To demonstrate the effect of compounds of formula I, which are enzymatically converted to 7-COOH-CBD once they enter the bloodstream, 3 month old Wistar rats, following acclimatization of 7 days prior to the start of the surgical procedure, were injected bilaterally with 3 mg/kg stz in the lateral ventricals and treated daily with either compound 1j (N═6; 25 mg/kg ip), 7-COOH-CBD (N=6; 20 mg/kg) or vehicle (N=6) for 8 consecutive days. On day 8, the animals were subjected to the novel object recognition (NOR) behavioural test. All objects used for the is test had similar textures, colors, and sizes but distinctive shapes. The rats were placed in a rectangular arena (76×49×57 cm) containing two identical objects (A1+A2) for the habituation and training phase. After 5 min, the rats were removed from the arena and placed in their home cage. After 24 h (long-term memory), the rats were placed in the arena again for 5 min, with one object replaced by a novel object (B+A2). Exploration of an object is defined as the time the animal spends with its head oriented towards the object, within two centimeters of the object (sniffing). The discrimination index was calculated by:

$$[ ( time exploring the new object ) / ( total time exploring the two objects ) ] \times 100 \%$$

### Results:

The novel object recognition test revealed a statistically significant worse long-term memory index for STZ animals, (49.8±12.1), when compared to STZ+7-COOH-CBD animals (79.6±3.4) or to STZ+example 1j (80.5±3.9). The novel object recognition test showed no difference in long-term memory index between animals treated with 7-COOH-CBD itself or with its cyclopropane ester.

1.-18. (canceled)

19. A compound of formula I, a diastereomer, enantiomer, crystal or co-crystal thereof, hydrate, solvate, or a pharmaceutically acceptable salt, or mixture thereof 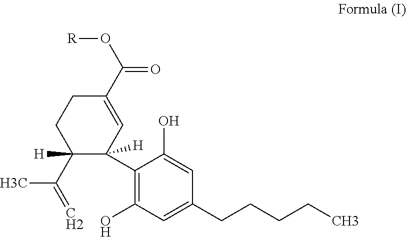
wherein R is selected from the group consisting of ethyl, propyl, isopropyl, propenyl, butyl, isobutyl, tertbutyl, butenyl, pentyl, pentenyl, cyclopropyl, cyclobutyl, cyclopentyl, cyclopentenyl, piperidinyl, piperidinomethyl, piperazinyl, piperazinylmethyl, piperazinylethyl, phenyl, phenylmethyl, phenylethyl, pyrrolinyl, pyrrolinylmethyl, pyrrolinylethyl, pyrrolinyl, pyrrolinylmethyl and pyrrolinylethyl, and R¹ is independently selected from the group consisting of fluoro, chloro, ═O, C(O)NR²R³, —COOR², R², —OR², and —NR²R³, wherein R² and R³ are independently selected from the group consisting of hydrogen and C₁₋₃alkyl.

20. The compound according to claim 19, wherein R is selected from the group consisting of ethyl, propyl, butyl, pentyl, hexyl, cyanomethyl, methoxymethyl, 2-oxoethyl, 2-aminoethyl, 2-cyanoethyl, 1-methylethyl, 2-methylethyl, 1,1-dimethylethyl, 2-cyano-1-methylethyl, 2,2-difluoroethyl, 2,2,2-trifluoroethyl, 2-(dimethylamino)ethyl, 2-amino-2-ethoxyethyl, 1-methylpropyl, 2-methylpropyl, 3-aminopropyl, 1,1-dimethylpropyl, 2,2-dimethylpropyl, 2-cyanopropyl, 3-(ethenyloxy)propyl, 3-oxobutyl, 1-methylbutyl, 2-methylbutyl, 3-methylbutyl, 1,1-dimethylbutyl, 2-propyn-1-yl, 2-propen-1-yl, 1-methyl-2-propyn-yl, 2-methyl-2-propen-1-yl, 3-buten-1-yl, 2-amino-1-methylethyl, 1-cyclopenten-1-yl, 3-cyclopenten-1-yl, cyclopropyl, 1-(hydroxymethyl)cyclopropyl, cyclobutyl, 3-hydroxy-cyclobutyl, 3-methoxy-cyclobutyl, 1-cyanocyclobutyl, cyclopentyl, 1-methylcyclopentyl, 3-methylcyclopentyl, 2-hydroxycyclopentyl, 3-hydroxycyclopentyl, 2,3-dihydroxycyclopentyl, cyclohexyl, 1Hpyrrol-2-yl, 1H-pyrrol-3-yl, 3-pyrrolidinyl, tetrahydro-3-furanyl, 3-pyridinyl, 2-thienyl, 1H-pyrrol-2-ylmethyl, 1H-pyrrol-3-ylmethyl, 2-furanylmethyl, 1H-imidazol-2-yl, 1-methyl-1H-pyrazol-3-yl, 5-methyl-2-furanyl, 1-cyclohexen-1-yl, 4-methyl-4H-1,2,4-triazol-3-yl, 1H-1,2,4-triazol-5-ylmethyl, 2-thiazolyl, 4-piperidinyl, 1-methyl-3-pyrrolidinyl, 1,3,4-thiadiazol-2-yl, phenyl, cyclopropylmethyl, cyclobutylmethyl, cyclopentylmethyl, (1-hydroxycyclobutyl)methyl, (i-hydroxycyclobutyl)methyl, (3-hydroxycyclobutyl)methyl, (3-methoxycyclobutyl)-methyl, (tetrahydro-2-furanyl)methyl, (tetrahydro-3-furanyl)methyl, 2-cyclopropylethyl, 1-cyclobutylethyl, 2-cyclobutylethyl, chlorophenyl, p-chlorophenylmethyl, metoxyphenyl, o-methoxyphenylmethyl, 2-aminocyclopentenyl, 2-fluorocyclopentenyl, piperidinomethyl, pyrrolinyl, pyrrolinylmethyl, pyrrolinylethyl, oxopyrrolinyl, oxopyrrolinylmethyl, piperazinyl, 1-piperazinyl)methyl, 4-methyl-1-piperazinyl)methyl and oxopyrrolinylethyl.

21. The compound according to claim 19, wherein R is selected from the group consisting of ethyl, propyl, isopropyl, n-butyl, 3-butenyl, 1-methylpropyl, isobutyl, tertbutyl, cyclopropane, cyclobutane, cyclopentane, 1-cyclopentene, 3-cyclopentene, 2-amino-1-cyclopentene and 2-fluoro-1-cyclopentene.

22. The compound according to claim 19, wherein R is selected from the group consisting of piperidinomethyl, 1-piperazinylmethyl, (4-methyl-1-piperazinyl)methyl, (p-chlorophenyl)methyl, (o-methoxyphenyl)methyl, and (5-oxo-3pyrrolin-2yl)methyl.

23. The compound according to claim 19, selected from the group consisting of
(3R-trans)-3-(2,6-dihydroxy-4-pentylphenyl)-4-(1-methylethenyl)-1-cyclohexene-1-carboxylic acid ethyl ester,
(3R-trans)-3-(2,6-dihydroxy-4-pentylphenyl)-4-(1-methylethenyl)-1-cyclohexene-1-carboxylic acid propyl ester,
(3R-trans)-3-(2,6-dihydroxy-4-pentylphenyl)-4-(1-methylethenyl)-1-cyclohexene-1-carboxylic acid isopropyl ester,
(3R-trans)-3-(2,6-dihydroxy-4-pentylphenyl)-4-(1-methylethenyl)-1-cyclohexene-1-carboxylic acid n-butyl ester,
(3R-trans)-3-(2,6-dihydroxy-4-pentylphenyl)-4-(1-methylethenyl)-1-cyclohexene-1-carboxylic acid 3-butenyl ester,
(3R-trans)-3-(2,6-dihydroxy-4-pentylphenyl)-4-(1-methylethenyl)-1-cyclohexene-1-carboxylic acid 1-methylpropyl ester,
(3R-trans)-3-(2,6-dihydroxy-4-pentylphenyl)-4-(1-methylethenyl)-1-cyclohexene-1-carboxylic acid isobutyl ester,
(3R-trans)-3-(2,6-dihydroxy-4-pentylphenyl)-4-(1-methylethenyl)-1-cyclohexene-1-carboxylic acid tertbutyl ester,
(3R-trans)-3-(2,6-dihydroxy-4-pentylphenyl)-4-(1-methylethenyl)-1-cyclohexene-1-carboxylic acid cyclopropane ester,
(3R-trans)-3-(2,6-dihydroxy-4-pentylphenyl)-4-(1-methylethenyl)-1-cyclohexene-1-carboxylic acid cyclobutane ester,
(3R-trans)-3-(2,6-dihydroxy-4-pentylphenyl)-4-(1-methylethenyl)-1-cyclohexene-1-carboxylic acid cyclopentane ester,
(3R-trans)-3-(2,6-dihydroxy-4-pentylphenyl)-4-(1-methylethenyl)-1-cyclohexene-1-carboxylic acid 1-cyclopentene ester,
(3R-trans)-3-(2,6-dihydroxy-4-pentylphenyl)-4-(1-methylethenyl)-1-cyclohexene-1-carboxylic acid 3-cyclopentene ester,
(3R-trans)-3-(2,6-dihydroxy-4-pentylphenyl)-4-(1-methylethenyl)-1-cyclohexene-1-carboxylic acid 2-amino-1-cyclopentene ester,
(3R-trans)-3-(2,6-dihydroxy-4-pentylphenyl)-4-(1-methylethenyl)-1-cyclohexene-1-carboxylic acid 2-fluoro-1-cyclopentene ester,
(3R-trans)-3-(2,6-dihydroxy-4-pentylphenyl)-4-(1-methylethenyl)-1-cyclohexene-1-carboxylic acid piperidinomethyl ester,
(3R-trans)-3-(2,6-dihydroxy-4-pentylphenyl)-4-(1-methylethenyl)-1-cyclohexene-1-carboxylic acid 1-piperazinylmethyl ester,
(3R-trans)-3-(2,6-dihydroxy-4-pentylphenyl)-4-(1-methylethenyl)-1-cyclohexene-1-carboxylic acid (4-methyl-1-piperazinyl)methyl ester,
(3R-trans)-3-(2,6-dihydroxy-4-pentylphenyl)-4-(1-methylethenyl)-1-cyclohexene-1-carboxylic acid (p-chlorophenyl)methyl ester,
(3R-trans)-3-(2,6-dihydroxy-4-pentylphenyl)-4-(1-methylethenyl)-1-cyclohexene-1-carboxylic acid (o-methoxyphenyl)methyl ester, and
(3R-trans)-3-(2,6-dihydroxy-4-pentylphenyl)-4-(1-methylethenyl)-1-cyclohexene-1-carboxylic acid (5-oxo-3pyrrolin-2yl)methyl ester.

24. A pharmaceutical composition comprising the compound of formula I, according to claim 19 in the association with a pharmaceutically acceptable adjuvant, diluent or carrier.

25. A pharmaceutical composition comprising (i) a compound of formula I, according to claim 19, and a pharmaceutically acceptable excipient, carrier or diluent, and (ii) at least one additional therapeutic agent, or a pharmaceutically acceptable salt thereof, and a pharmaceutically acceptable excipient, carrier or diluent.

26. A method of preventing, progression prophylaxis, or treating a disease of the central nervous system in a subject comprising administering to the subject a compound of claim 19.

27. A method of preventing, progression prophylaxis, or treating a disease or disorder selected from the group consisting of Alzheimer disease (AD), Parkinson's disease (PD), Lewy Body disease (LBD), Lou Gehrig's disease (ALS), Huntingdon's disease, and frontotemporal dementia (FTD) in a subject comprising administering to the subject a compound of claim 19.

28. A method of preventing, progression prophylaxis, or treating a neurodegenerative disease or disorder in a subject comprising administering to the subject a compound of claim 19.

29. A method of preventing, progression prophylaxis, or treating Alzheimer disease (AD) in a subject comprising administering to the subject a compound of claim 19.

30. A process for manufacturing compounds of formula I, as defined in claim 19, comprising
reacting R-1-imidazolecarboxylate (R-ImC) with 7-COOH-CBD in a polar solvent at a temperature of 20 to 80° C. for 10 to 40 hours under nitrogen atmosphere, to obtain the compound of formula I.

31. A process for manufacturing compounds of formula I, as defined in claim 19, comprising the steps of
1a) reacting imidazole and R-chloroformate in a suitable organic solvent to obtain R-ImC, and
1b) reacting R-1-imidazolecarboxylate (R-ImC) with 7-COOH-CBD in a suitable polar solvent at a temperature of 20 to 80° C. for 10 to 40 hours under nitrogen atmosphere, to obtain a compound of formula I.

32. The process according to claim 31, wherein the organic solvent in step 1a) is selected from the group comprising dichloromethane, 1,2-dichloromethane, acetonitrile, tetrahydrofuran, 2-methyl tetrahydrofuran and 1,2-dimethoxy ethane.

33. The process according to claim 31, wherein the polar solvent is selected from the group comprising acetonitrile, tetrahydrofuran, 2-methyl tetrahydrofuran, N,N-dimethyl formamide, dichloromethane and 1,2 dichloro-ethane, or any mixture thereof.

34. The process according to claim 31, wherein the molar ratio of 7-COOH-CBD to R-1-imidazolecarboxylate (R-ImC) is 0.25 to 2:1 to 3, or 1:2.
